(19) United States
(12) Patent Application Publication
(10) Pub. No.: US 2003/0049659 A1
(43) Pub. Date: Mar. 13, 2003

(54) Devices and methods for isolating samples into subsamples for analysis

(76) Inventors: Stanley N. Lapidus, Bedford, NH (US); Walker M. Sloan III, Berlin, MA (US)

Correspondence Address: TESTA, HURWITZ & THIBEAULT, LLP, HIGH STREET TOWER, 125 HIGH STREET, BOSTON, MA, 02110, US

(21) Appl. No.: 10/158,572

(22) Filed: May 29, 2002

Related U.S. Application Data
(60) Provisional application No. 60/294,323, filed on May 29, 2001.

(51) Int. Cl. C12Q 1/68

(52) U.S. Cl. 435/6; 422/101

(57) ABSTRACT

The invention provides devices and methods for analyzing a sample by distributing the sample along a continuous channel provided by a pathway of concatenated wells. The concatenated wells can be isolated from each other to form a series of independent subsamples. The invention is especially useful to isolate entities suspected to be present in a sample for individual analysis.

### CROSS-REFERENCE TO RELATED APPLICATIONS

This application claims the benefit of and priority to U.S.S.N. 60/294,323, filed May 29, 2001, the disclosure of which is incorporated by reference herein.

### FIELD OF THE INVENTION

The invention relates to devices and methods for distributing a liquid sample along a continuous pathway provided by concatenated wells. Liquid sample is distributed along the pathway and isolated within individual wells prior to its analysis.

### BACKGROUND OF THE INVENTION

The ability of a scientist or a researcher to analyze samples in microscopic amounts is of paramount importance, both in terms of efficiency and specificity, to the biomedical community. For example, information obtained from the analysis of nucleic acids provides scientists and researchers with indispensable understanding of life processes and biological systems. Knowledge gained from the analysis of certain nucleic acids provides researchers with insight into the relationship of various components in biological processes such as the function of particular proteins in signaling pathways that may aid medical professionals to properly diagnose and treat patients with certain genetic disorders.

The study of nucleic acids allows researchers to identify causes of diseases or disorders that involve mutations, insertions, deletions or repeats of certain portions of the genome. Detection of the presence of one or more molecules present in low-frequency in a complex biological sample is of special interest for many researchers and clinicians focused on the detection and treatment of disease. As progress is made with non-invasive or minimally invasive methods of treatment such as, for example, the analysis of specimens such as stool, sputum, and other biological samples having a complex mixture of cellular components, the ability to detect mutations in this complex environment may be the determining factor in the early detection of a given disease.

For example, generally, the earlier a patient is diagnosed with cancer, the greater the likelihood there exists a more effective treatment of the disease. Therefore, the detection of mutations in oncogenes or tumor suppressor genes, or the detection of loss of heterozygosity (LOH) in tumor suppressor genes at an early stage of oncogenesis may present a pivotal advantage in the successful treatment or prevention of disease. For example, DNA from cells having mutations indicative of early-stage cancer are present in complex specimens containing cellular components in low frequency with respect to wild-type DNA. Therefore, detection of mutant DNA in a sample using conventional techniques is often difficult even if the target DNA (potentially containing mutant DNA) is present in the specimen, or in a sample derived from the specimen. Quite often, manual or substantially manual techniques fail to adequately obtain and detect target polynucleotides that exist in low frequency.

Similar problems exist in the detection of other low-frequency molecular species. For example, the detection of the relative amounts of high- and low-expression proteins may be undetectable over highly-expressed proteins. A similar situation exists when detecting RNA. In addition to nucleic acids, proteins and RNA, the lack of success in detecting low-frequency molecular species is a problem for other cellular components as well.

Current genetic methods are generally capable of detecting nucleic acids more routinely. For example, Polymerase Chain Reaction (PCR) allows a person to amplify substantial amounts of a polynucleotide of interest. However, PCR alone does not effectively resolve the problem of detecting low-frequency molecules in a heterogenous, complex sample. For example, PCR hypothetically amplifies a low-frequency nucleic acid only if the PCR primers hybridize to the low-frequency sequence. However, if a low-frequency target amplicon is not amplified in the first rounds of PCR, the probability that it will be amplifiable and detectable in successive rounds decreases with each round. As a result, attention must be paid to the amount of material presented to the PCR, the efficiency of the PCR, and the representative nature of the input sample. See, e.g. PCT International Publication No.: WO 00/32820. This, and other genetic methods, rely on a multiplicity of distinct processes to identify nucleic acid sequences, which introduces a potential for error in the overall process.

Various devices and methods have been developed to overcome the limitations of using traditional PCR techniques to detect low-frequency polynucleotides. See, Vogelstein et al., Proc. Natl. Sci., 96: 9236-9241 (1999), incorporated by reference herein. Digital PCR results in amplification of single, target molecules to produce a digital signal that is especially good for detecting low-frequency polynucleotides. Specifically, digital PCR operates by taking a sample, diluting it, and dividing it into tens of thousands of subsamples, each one in its own well, so that most subsamples contain either zero or one target polynucleotide(s), and very rarely do subsamples contain more than one target. The subsamples are then amplified and detected individually using PCR. PCR performed on subsamples results in pure amplification of a single polynucleotide, whether mutant or wild-type. Consequently, each well or discrete result in the PCR is a homogeneous replicate of the original single-starting polynucleotide. This makes possible the determination of whether that starting polynucleotide was mutant or wild-type. The entire sample can then be characterized simply by counting the number of mutant and wild-type wells and taking the ratio.

Notwithstanding the progress made with Digital PCR, there are still limitations with the ability to distribute and fractionate sample among numerous wells appropriate for the detection of low-frequency polynucleotides. Similar problems exist if the sample has been pooled from different patients where target nucleic acids exist in the pooled sample in low frequency compared to other nucleic acids.

Moreover, technological advances in the semiconductor industry have been capitalized with the development of certain micro-mechanical fluidic devices that contain, for example, miniature pumps, valves, reservoirs and passageways to meet the needs of researchers seeking effective solutions for detecting low-frequency events. Companies that have rushed to market with various micro-analytic devices have not only failed to adequately develop devices to effectively detect these low-frequency events, but the companies have also neglected to adequately prevent problems associated with PCR that may distort the analysis as a result of contamination, evaporation and/or leakage, for example. It is common for leakage, for example, to result in preferential amplification of a specific product that may or may not contaminate the assay by producing a signal that could be a false positive or negative.

Accordingly, there is a need in the art for devices and methods for efficiently detecting and analyzing target nucleic acids with confidence by distributing and isolating the nucleic acids prior to their amplification and analysis. At the same time, such devices and methods must effectively prevent contamination, evaporation and leakage while allowing for fractionation without the need for pipetting or other non-efficient manual techniques.

### SUMMARY OF THE INVENTION

The present invention generally provides devices and methods for conducting parallel and serial reactions on subsamples or fractions of material in a sample solution. Such subsamples may include one or more biological molecule or molecules (e.g., a nucleic acid, a protein, a carbohydrate, etc.), cellular components, by-products of cellular metabolism, cellular debris, whole cells, or acellular molecules (e.g. minerals, metals, etc.) or a combination of the above. Thus, as referred to herein, the term “material” refers to any cell, cellular component, or noncellular substrate the detection and/or reaction of which is desired. A sample for testing may be from any source that can be dissolved or extracted into a liquid, and which may potentially contain one or more of the analytes of interest. A preferred embodiment of the sample may be a biological solid or fluid such as, for example, blood, stool, serum, plasma, urine, sweat, tear fluid, saliva, semen, cerebral spinal fluid, or a purified or modified derivative therefrom.

A preferred embodiment of the invention includes the distribution, isolation and analysis of samples including cells. For example, cells obtained with a Pap smear may be distributed, isolated, and may also be analyzed. More specifically, embodiments of the invention provide for the distribution and isolation of cellular material obtained from a human, animal or plant, for example, the cellular material obtained from the uterine cervix. In a preferred embodiment, individual cells are isolated and analyzed in separate sample chambers or wells. The isolation of cells in individual wells provides for a greater likelihood of detecting the presence or absence of disease such as, for example, cervical cancer, precancerous lesions and a variety of other related infectious and disease conditions. The isolation of cells into individual wells decreases sampling errors as well as screening errors commonly associated with the detection and analysis of cells via conventional slide evaluation. Sampling and screening errors are prevalent with the analysis of cellular material, such as those obtained, for example, from the uterine cervix, because of the difficulty of analyzing overlapping layers of cells on a slide. Other embodiments of the invention include the analysis of components of cellular material that also benefits from individual analysis.

The invention provides micro-fluidic devices and methods for their use which allow for fractionation of a liquid sample. According to the invention, a sample fractionation device includes, generally, a first layer slidably attached to a second layer, each layer having at least one well. The device comprises at least two wells with at least one well in each respective layer. In one embodiment, the invention provides for a device having a first position wherein a first well from one layer of the device is in fluid communication with a second well from the second layer of the device. In a preferred embodiment, the first and second layer each include a plurality of wells as shown, for example, in FIGS. 1 and 2. The first and second layers include individual wells arranged in series, which are designed to maximize the number of wells in a pre-determined surface area on a substrate. For example, as shown in FIG. 3, one embodiment of the invention provides for a device having both the first and second layers in a first position whereby a series of concatenated wells forms a continuous channel or pathway allowing for the distribution of a sample solution. In a preferred embodiment of the device, a series of wells creates a network or pathway for the distribution of sample solution to a multiplicity of wells. Also, other embodiments of the invention further provide more than one series of networks or pathways for the distribution of sample solution along a multiplicity of concatenated well networks. As shown, for example, in FIG. 4, the device also has a second position wherein the individual wells are isolated. In one embodiment, a shift or slide of either the first or second layer of the device disconnects the plurality of once-concatenated wells into individual wells.

Also as shown, a preferred embodiment provides for a device having at least two concatenated well networks or pathways. In a related embodiment of the invention, a device has a first position providing at least one concatenated well network or pathway for PCR brew including PCR reagents (e.g. buffer, nucleotides, thermostable polymerase, and primers), and at least one concatenated well network or pathway for molecular beacons or other polynucleotides that preferably can be detected. The device has a second position, wherein the wells in the once-concatenated well networks or pathways are isolated. In embodiments of the invention wherein detection and/or analysis of nucleic acids are performed, the second position allows for the performance of amplification with the device by thermocycling, for example. In a further related embodiment, the device also has a third position wherein an individual isolated well containing the PCR brew having a nucleic acid(s) and an individual isolated well containing molecular beacons or other polynucleotides are overlapped. In embodiments of the invention wherein detection and/or analysis of nucleic acids are performed, the third position allows for the mixing of the PCR brew containing the nucleic acid(s) with molecular beacons or other polynucleotides. Mixing of the PCR brew and the molecular beacons or other polynucleotides may be performed, for example, by raising and lowering temperature or by immersing the plates in an ultrasonic bath.

The invention solves the problem of sample fractionation (distribution of a single sample into a multiplicity of subsamples), especially when it is desired to isolate low-frequency material thought to be present in the sample or when the required subsample volume is too small to allow effective serial dispensing using physical processes such as pipetting.

In a preferred embodiment, a sample solution is divided into a plurality of subsamples in a process that results in the discrete distribution of material for analysis and/or detection. According to the one embodiment of the invention, a sample moves through a device—the device having a first position where a first well is in fluidic communication with a second well. In a preferred embodiment, at least two wells in fluidic communication are linked together in a concatenated position when the device is in a first position. This allows a sample solution to flow from a first well to at least a second well arranged in series. After a pre-determined time or a predetermined amount of sample solution has flowed through the concatenated wells, sample solution is isolated into discrete wells after the device is moved to the second position—each well no longer in fluidic communication with the previous- or after-adjoining well or any other well.

In a preferred embodiment, devices and methods of the invention provide for the isolation and analysis of at least one molecule in each individual well when the device is in the second position. Generally, the invention operates to isolate small numbers of molecules or nucleic acids for detection, identification and analysis. Target and non-target molecules or nucleic acids will be distributed in very small volumes according to the laws of probability. Thus, in a preferred embodiment, most wells will have no target molecules or nucleic acids, some will have one, and very few will have more than one. The presence of substantially one molecule in individual wells provides for the digital analysis of molecules or nucleic acids as provided for in the methods and devices of the present invention. In another embodiment, each well has on average 5 to 10 target nucleic and molecules after subsample isolation and prior to amplification.

In another preferred embodiment, sample solution is forced from a first well to a next well, and so on, by a pressure source that induces the flow of sample solution. Increased pressure causes sample solution to flow through the concatenated wells while the device is in the first position. When the device is in the second position, the force created by the pressure source is inhibited due to the fact the wells are no longer connected. In a preferred embodiment of the invention, a sample suction port (40) is connected to the last well or distally in a series of concatenated wells when the device is in the first position and serves as the pressure source thereby allowing for the introduction of a vacuum as shown, for example, in FIGS. 2, 3, and 4. In a highly preferred embodiment of the invention, air is pre-evacuated from a sample suction port creating a vacuum in the pathway of concatenated wells prior to the introduction of sample in the fill port (20) as shown, for example, in FIGS. 1, 3, and 4. Also, in an embodiment of the invention, sample solution is prevented from flowing out of the device from the distal end of the well pathway by a hydrophobic valve which actuates at a higher pressure.

A preferred device of the invention includes a reservoir for holding a sample solution comprising sample and any reagents such as, for example, PCR components. In a preferred embodiment, the reservoir is in fluid communication with the first well in the pathway or network of wells into which a portion of the sample solution flows. In operation, sample solution including the sample (and any reagents) is deposited into the reservoir and introduced into the device through the network or pathway of wells. Thereafter, the device is moved into the second position, wherein each well is isolated and contains a subsample. In a particularly preferred embodiment of the invention, each of concatenated wells in the pathway or network are filled completely prior to the time the device is moved to the second position.

A preferred device of the invention includes a vacuum system having at least one vacuum line that secures the two layers together when the device is in either the first position, the second position, and during the time the device is moved from the first to the second position. Also, in preferred embodiments of the invention, a vacuum port (50) is connected to the vacuum line providing the vacuum or pressure necessary to secure the two layers together as shown in FIGS. 1, 3, and 4. In other embodiments of the invention, devices include additional vacuum lines having separate or shared vacuum port(s) that serve to increase the attraction between the two layers. Also, the two layers may be secured while the device is in the first position, the second position or during the time the device is moved from the first to the second position by external forces. For example, orthogonal pressure or substantially orthogonal pressure may be provided on the opposing sides of the two layers by any mechanism or force that provides or maintains an appropriate amount of external pressure to secure the two layers. Also, in a preferred embodiment, the two layers of the device are lubricated or lubed with mineral oil or other lubricant promoting a tight seal between the two layers when the plates are aligned together.

In a preferred embodiment, the device further comprises a fill port for introducing the sample solution to be analyzed into the device. When the device is in the first position, the fill port is connected to the first well in the pathway or network of concatenated wells allowing for the introduction of sample solution to the device. In other embodiments, additional pathways or networks may have separate or shared fill port(s).

In an embodiment, the components of the device including for example, the wells, vacuum lines, and ports are etched, machined, stamped, or embossed onto a substrate. As discussed herein, the invention further provides an efficient design that allows for cost-effective and scalable manufacture and assembly of such micro-fluidic devices and methods for their use. Namely, a preferred embodiment of the invention provides for a device having identical or symmetrical first and second layers. In operation, the second layer is rotated 180 degrees, or “flipped over”, with respect to the first layer (or vice versa—the first layer rotated 180 degrees with respect to the second layer). In addition to the obvious manufacturing efficiency and related cost-savings provided by the design, the identical design embodiment may provide purchasers of the product with low-cost alternatives to the replacement of damaged devices by enabling purchasers to obtain single-layer replacements instead of non-bifurcated products.

The present invention also provides methods of separating a sample solution by controlling and manipulating an embodiment of a device disclosed herein. Generally, the invention provides methods for separating a sample solution in a device comprising a first layer slidably attached to a second layer. The device and its components, including the relationship between the components are described herein. In one embodiment of the invention, a method includes sliding a device from a first position (where the wells are in fluidic communication) to a second position, thereby separating the sample within the individual wells.

The present invention also provides methods of applying a pressure source in the device thereby actuating movement of the liquid sample. In a preferred embodiment, the pressure source is presented to the first well in the pathway or network of concatenated wells while the device is in the first position. As described herein, when the device is in the second position, the force created by the pressure source is inhibited due to the fact the wells are no longer connected. Alternatively, the pressure source is removed when the device is switched from the first to the second position. In other embodiments, as described herein, methods of the invention include introducing a sample solution into the device through the fill port after air has been pre-evacuated by a pressure source via the sample suction port while the device is in the first position as shown in FIG. 3.

In other embodiments, methods of the invention further provide for the use of molecular beacons or other nucleic acids to detect target nucleic acids to facilitate the analysis steps. An embodiment of the invention further includes the use of labels on a target nucleic acid sequence with a detectable label, such as, for example, radioisotopes, fluorescent compounds, and enzymatic markers.

After amplification such as, for example, by polymerase chain reaction, the invention further provides for the analysis of subsamples present in the isolated wells. In a preferred embodiment, the invention includes the determination of whether a difference exists between the amounts of a first target nucleic acid and a second target nucleic acid. The presence of a statistically-significant difference being indicative of a presence of disease in a patient from whom said sample is obtained.

For devices having at least two concatenated well networks or pathways, methods of the invention provide for introducing liquid sample containing PCR brew in at least one concatenated well network or pathway, and introducing molecular beacons or other polynucleotides (that preferably can be detected) in at least one other concatenated well network or pathway. In a related embodiment, methods of the invention provide for the isolation of the wells containing PCR brew, molecular beacons or other polynucleotides. Such isolation methods involve sliding the device or layer from the first position to a second position. Also, in a related embodiment, methods of the invention provide for mixing the contents of a well having PCR brew with a well having molecular beacons or other polynucleotides by moving the device or layer from the second position to a third position.

For devices having at least two concatenated well networks or pathways where at least one network or pathway contains PCR brew and at least one network or pathway contains molecular beacons or other polynucleotides, methods of the invention provide for amplifying PCR brew by thermocycling. Amplification may be conducted after sliding a layer from the first position to the second position (or when the wells are isolated or separated having a homogeneous content). In a further related embodiment of the invention, after the device is moved from the second position to the third position such that an individual isolated well contains both PCR brew and molecular beacons (or other polynucleotides), methods of the invention provide for mixing the PCR brew and the molecular beacons (or other polynucleotides) by raising and lowering the temperature. In further embodiments of the invention, mixing the PCR brew and the molecular beacons (or other polynucleotides) is performed by immersing the plates in an ultrasonic bath.

In additional embodiments, after the detection and/or analysis of the nucleic acids, the invention includes the steps of performing a colonoscopy or a sigmoidoscopy on a patient from whom the sample solution is obtained.

The device of the invention may also be provided as part of a kit which may additionally include, for example, selected reagents, sample preparation materials, and instructions for using the device. The kit may also include instructions describing all, or part of, the methods of the invention as disclosed herein.

In another aspect of the invention, embodiments provide for a fluidic switch manipulated by slidable layers of concatenated wells allowing for the joining (and disjoining) of circuits and/or pathways. Embodiments of the invention may be used for detecting and analyzing nucleic acids. Additional embodiments of the invention, may be used with devices used for conducting reactions, such as specific assays, for example, a DNA integrity assay, a Bat-26 assay, an assay to detect loss of heterozygosity, and the like. See e.g., U.S. Pat. Nos. 5,670,325 and 6,143,529, and U.S.S.N. 09/455,950 and U.S.S.N. 09/940,225, the disclosure of each of which is incorporated by reference herein.

### BRIEF DESCRIPTION OF THE DRAWINGS

The advantages of the invention may be better understood by referring to the description herein taken in conjunction with the accompanying drawings. The drawings are not necessarily to scale, emphasis instead being placed on illustrating the principles and concepts of the invention in which:

FIG. 1 is a schematic diagram of a lower layer of an exemplary device.

FIG. 2 is a schematic diagram of an upper layer of an exemplary device.

FIG. 3 is a schematic diagram of an exemplary device in the first position showing the concatenated wells forming a continuous channel.

FIG. 4 is a schematic diagram of an exemplary device in the second position showing isolated wells.

### DETAILED DESCRIPTION OF THE INVENTION

It is the general object of the present invention to provide micro-fluidic devices and methods for distributing a sample solution along a continuous pathway or network provided by concatenated wells whereby molecules in solution are distributed and isolated within individual wells prior to analysis. Devices and methods are useful in a number of applications, for example, with the detection and analysis of nucleic acids and various other sequencing applications. Particularly, the invention solves the problem of sample fractionation (distribution of a single sample into a multiplicity of subsamples), especially when it is desired to isolate low-frequency material thought to be present in the sample or when the required subsample volume is too small to allow effective serial dispensing using physical processes such as pipetting.

Skilled artisans practicing in these fields generally will understand that, notwithstanding manual detection and analysis and the interpretation thereof, the invention provided herein may be a part of a larger system which includes, for example, automated detection and analysis such as with robotics. For example, the data collected from the devices and methods provided herein may be interpreted using automated or computer-enabled machines such as, for example, optical readers for efficient analysis of the subsamples.

Generally, the present invention provides devices and methods for conducting parallel reactions on material in a sample solution. As described in detail herein, such material may be one or more molecules for which the detection and/or reaction of which is desired. In operation, the invention generally comprises micro-fluidic devices and methods for their use which allow for fractionation of a sample solution. According to the invention, a sample fractionation device comprises a first layer attached to a second layer. The device has a first position wherein at least two wells are in fluidic communication, and a second position wherein the two wells are isolated from each other. The device and the operation thereof provides for the identification, amplification and/or analysis of a sample in solution, while preventing contamination of the sample by external and internal contaminants, or contamination of the environment by the sample.

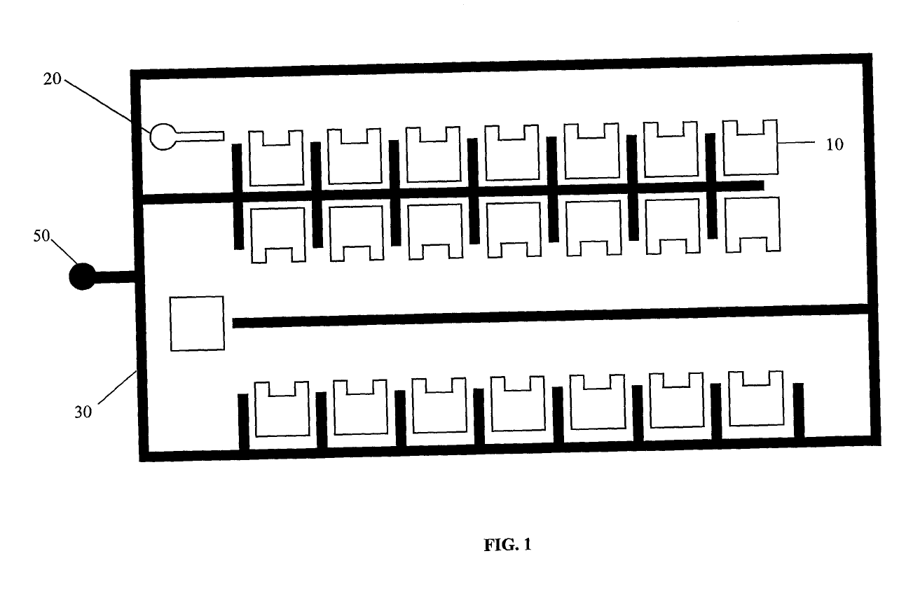

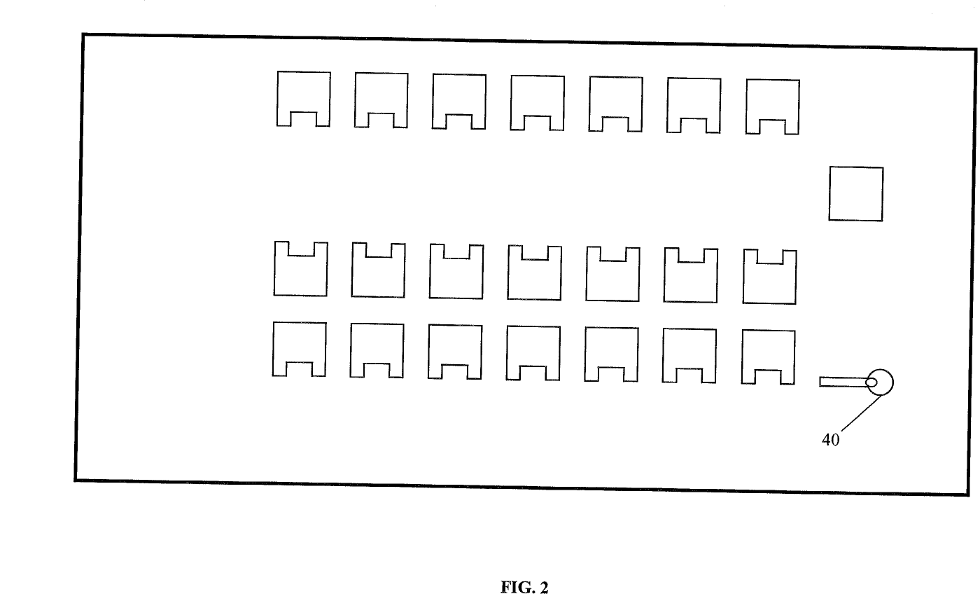

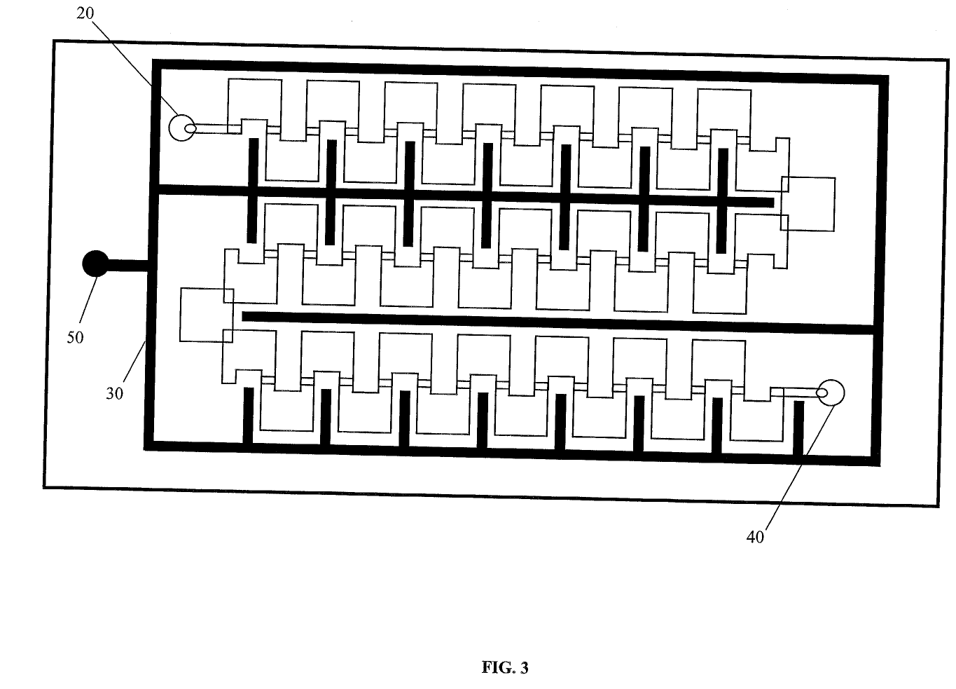

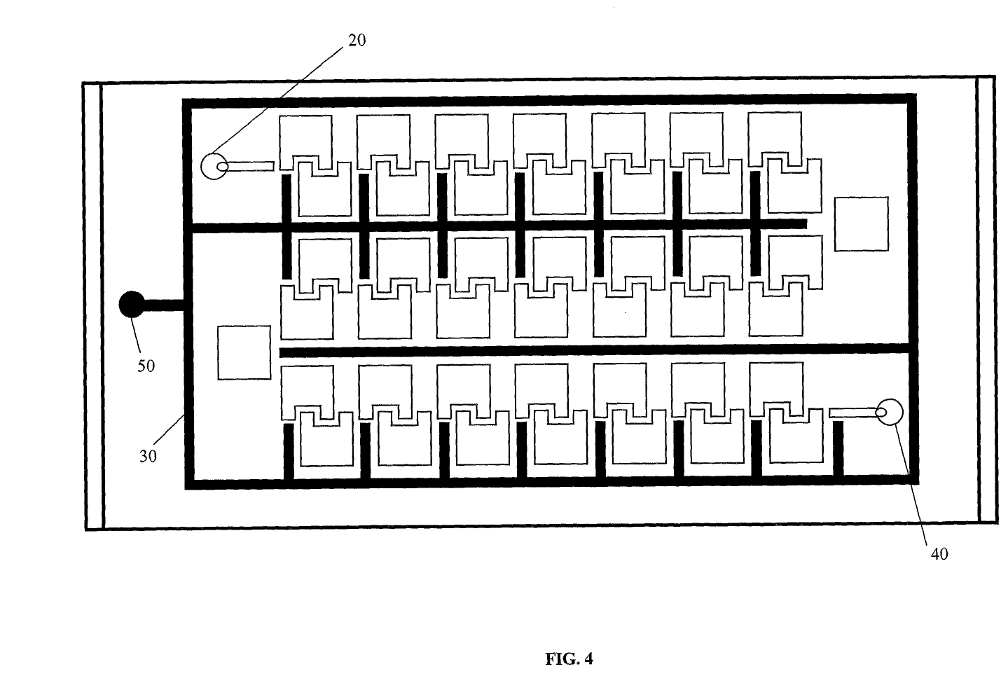

According to the invention, a device includes a first layer with at least one first well and a second layer with at least one second well. The first and second layers are slidable with respect to each other. When the two layers are in a first position with respect to each other, a well in the first layer overlaps with a well in the second layer to form a fluidic channel connecting the two wells. When the two layers are in a second position with respect to each other, the well in the first layer is isolated from the well in the second layer. As described herein, a preferred embodiment of the device comprises a series of wells that form a network or pathway for the distribution of sample solution to a multiplicity of wells when the device is in the first position. Each layer includes a plurality of wells, arranged such that successive wells in each layer overlap when the device is in the first position, thereby forming a continuous channel of concatenated wells (as shown for example in FIG. 3). For example, in the first position, a first well in the first layer overlaps with a second well in the second layer. This second well also overlaps with a third well in the first layer that in turn also overlaps with a fourth well in the second layer, and so on to form a continuous channel involving successive wells alternating between the two layers. According to the invention, the network or pathway is disrupted when the device is in the second position and each well is isolated (as shown for example in FIG. 4). Also, other preferred embodiments of the invention further provide for more than one parallel network or pathway for the distribution of sample solution thereby allowing for the isolation of molecules such as nucleic acids along a multiplicity of concatenated well networks. The design of the invention allows for the number of wells in a pre-determined substrate to be maximized. In a preferred configuration, such as the configuration shown in FIGS. 1-4, the wells are directly connected to each other in the first position, without using additional connecting or filling lines. The lack of extra fill lines allows for a high density of wells in a substrate. For example, a preferred embodiment of the invention may provide approximately 25,000 to 100,000 wells in the dimensions of a standard 96 well plate.

An advantage of using more than one network or pathway of concatenated wells for the distribution (and later isolation) of sample is that the time needed to fill the multiplicity of wells may be significantly reduced relative to the time required to fill the same number of wells using a single network or pathway of concatenated wells.

In a preferred embodiment, a sample solution is divided into a plurality of subsamples in a process that results in the discrete distribution of material for analysis and/or detection. According to the invention, a sample solution moves through a device—the device having a first position where a first well is in fluidic communication with a second well. In a preferred embodiment, at least two concatenated wells in fluidic communication allow a sample solution to move from a first well to at least a second well. After a pre-determined time or after a predetermined amount of sample solution has flowed through the concatenated wells (or once the wells are filled), sample solution is isolated into discrete wells in the device by switching the device to the second position, where the wells are no longer in fluidic communication.

In a particularly preferred embodiment, sample solution is forced from one well to a second contiguous well, and so on, by a pressure source that induces the flow of sample solution. Increased pressure causes sample solution to flow through the concatenated wells while the device is in the first position. When the device is in the second position, the force created by the pressure source is inhibited due to the fact the wells are no longer contiguous. For example, in a highly preferred embodiment of the invention, the pressure system provides for the pre-evacuation of air (creating a vacuum) inside the wells prior to the introduction of sample into the device. Also, in one embodiment of the invention, sample solution is prevented from flowing out of the device from the last well by a hydrophobic valve which actuates at a higher pressure. In another embodiment, positive pressure may be provided when the wells are in fluidic communication. Positive pressure may be provided, for example, at the fill port, to cause the sample to flow through the concatenated wells.

A preferred device of the invention comprises a reservoir for holding a sample solution comprising sample and any appropriate reagents, such as, for example, molecular beacons, markers and PCR components. The reservoir is in fluid communication with the concatenated well(s) into which a portion of the sample solution flows. In operation, sample solution comprising the sample is deposited into the reservoir and is forced into the device through the pathway or network of concatenated wells (while the device is in the first position). Thereafter, the device is moved into the second position, thereby isolating a sample fraction or subsample into each well.

The invention further provides devices and methods for its use that resolve the problem of leakage and evaporation often associated with prior art devices and methods. For example, as shown in FIGS. 1, 3 and 4, a preferred device of the invention comprises a plurality of wells (10), and a vacuum system (30) including at least one vacuum line that secures the two layers together when the device is in either the first position, the second position, and during the time the device is moved from the first to the second position. Also, in preferred embodiments of the invention, a vacuum port (50) is connected to the vacuum line providing the vacuum or pressure necessary to secure the two layers together as shown in FIGS. 1, 3, and 4. In other embodiments of the invention, devices include additional vacuum lines having one or more separate or shared vacuum port(s) that serve to increase the attraction between the two layers. The larger the surface area covered by the vacuum system, the greater the attraction between the first and second layers of the device. Embodiments of the invention provide for the reduction or cessation of vacuum in the vacuum system during the time the actual sliding motion is taking place.

Also, the two layers may be secured while the device is in the first position, the second position or during the time the device is moved from the first to the second position by external forces. For example, orthogonal pressure or substantially orthogonal pressure may be provided on the opposing sides of the two layers by any mechanism or force that provides or maintains an appropriate amount of external pressure to secure the two layers.

Also, in a preferred embodiment, the two layers of the device are lubricated or lubed with mineral oil or other lubricant promoting a tight seal between the two layers when the plates are aligned together.

In a preferred embodiment, the two layers of the device are lubricated or contacted with mineral oil or other lubricant that promotes a tight seal between the two layers when the plates are aligned together. In other embodiments, the lubricant or mineral oil may be applied by silk screening onto the respective layer directly using, for example, a mask to keep the oil from contaminating the wells or vacuum lines. Also, the lubricant or mineral oil may be applied by contacting the inner surface of at least one of the two layers of a device to a mineral oil layer which has been silk screened onto a plain sheet of glass, thereby not requiring a mask. In addition, embodiments of the invention include the use of an alcohol with the mineral oil that facilitates its application to the device. Generally, mineral oil or any other appropriate hydrophobic substance provides the necessary lubrication, and also does not affect the later amplification and analysis steps for the sample.

The device also provides a solution to the common issue of evaporation when conducting PCR (including PCR involving micro-analytic devices). By having a concatenated well system that results in the complete filling and isolation of each individual well prior to PCR, there is no concern about evaporation, and therefore a “hot bonnet” PCR is not necessary.

The invention further provides an efficient design that allows cost-effective and scalable manufacturing and assembly of such micro-fluidic devices and methods for its use. Namely, a preferred embodiment of the invention provides for a device having identical or symmetrical first and second layers with the second layer rotated 180 degrees with respect to the first layer, or vice versa (first layer rotated 180 degrees with respect to the second layer).

The substrate that defines the network or networks of concatenated wells and the other components, such as, for example, the vacuum network, may be formed from any material that is suitable for conducting analyte detection. Materials that may be used include, for example, various plastic polymers and copolymers, such as, polypropylenes, polystyrenes, polyimides, and polycarbonates. Inorganic materials such as glass and silicon are suitable as well. Also, the substrate may be formed from one or more materials.

The opacity or transparency of the device, as well as the depth of each layer's wells, will generally influence the appropriate characteristics of the detecting mechanism. Due to the fact that the device is composed of two layers, each layer having wells disposed therein, embodiments of the invention provide for various optical transparencies for efficient detection mechanisms. For example, for fluorescence detection, the opaque substrate material preferably exhibits low reflectance properties so that reflection of the illuminating light back toward the detector is minimized. Conversely, a high reflectance may be desirable for detection based on light absorption. Appropriate detection criteria are well known to skilled artisans practicing in the art of analyte detection and analysis.

The wells in the device are designed generally to be as miniature as possible, in order to achieve a high density of wells per device. In a highly preferred embodiment of the invention, the wells in each layer of the device are etched with a depth that is nominally about 100 μm to 200 μm. In other embodiments, the wells in each layer of the device can be less than 100 μm or greater than 200 μm. Generally, the size of the wells depends on factors, including, for example: the focal requirements of readers such as optical readers; the type of detectable labels that are used such as, for example, radioisotopes, fluorescent compounds, and enzymatic markers; the material to be distributed, isolated and analyzed; and, the requirements of the end-user for proper manipulation of the device. For example, individual wells may have a cross-section or a diameter from about 10 μm to about 1 mm and preferably from about 100 μm to 500 μm. Also, in preferred embodiments of the invention, the wells and other components in the device are etched on a substrate in the manufacturing process. In other embodiments, the device of the invention contemplates the components having been machined, stamped or embossed on a substrate in the manufacturing process, such as, for example, with a laser.

Although the figures in the attached drawings show chambers having a square or rectangularly shaped overhead cross-section, other geometries, such as diamonds, circles, ovals, irregularly shaped, or a combination of the above, may also be used. Preferred well or chamber volumes range from about 1 to 10 μl to about 1 to 10 nl. However, larger or smaller volumes may also be used. Similarly, channels for the vacuum pathway, as well as the sample supply ports and sample suction ports may be straight or curved, as necessary, with cross-sections that are shallow, deep, square, rectangular, concave, or V-shaped, or any other appropriate configuration. In a preferred embodiment, a well or chamber includes one or two extensions (as shown in FIGS. 1-4) such that the extensions of successive wells in the first and second layers overlap when the device is in the first position (shown in FIG. 3) to form a continuous channel of concatenated wells.

In a preferred embodiment, the invention provides for the amplification of a nucleic acid using polymerase chain reaction (PCR). Once a sample is loaded onto the device (along with necessary PCR buffers, nucleotides and enzymes) and fractionated as described herein, it is exposed to appropriate conditions for PCR amplification. The presence or absence of a PCR product in each well or chamber is preferably determined using a fluorescence-based detection method. Specifically, a preferred method uses molecular beacons. Molecular beacons are dual-labeled oligonucleotides having a reporter at one end and a quencher at the other end. The oligonucleotide is further designed such that in the absence of target the oligonucleotide forms a hairpin structure that brings the reporter and quencher in physical proximity resulting in efficient quenching of the reporter. However, in the presence of a complementary target sequence, the probe molecule unfolds and hybridizes resulting in a physical separation of the reporter and quencher groups. As a result, the reporter will emit a signal upon stimulation. The functions and characteristics of molecular beacons are known to artisans skilled in the art. Other embodiments of the invention provide for the determination of product using UV fluorescence with ethidium bromide.

Alternatively, samples may be analyzed using, for example, one or more known enzymatic or chromographic based assays. According to a preferred embodiment of the invention, a sample is mixed with an appropriate assay buffer before being loaded into the device. Accordingly, each subsample contains all the components for the detection assay. In another highly preferred embodiment of the invention, appropriate assay buffers and/or PCR reagents are loaded into the device by a separate pathway or network of concatenated wells that mix with sample solution while the device is moved from the first to the second position. In a highly preferred embodiment of the invention, appropriate assay buffers and/or PCR reagents are mixed with sample solution in an intermediary step between the first and second positions described herein. In another highly preferred embodiment of the invention, appropriate buffers and/or PCR reagents are mixed with sample solution in a step following the second step of isolating the sample into subsamples.

The invention further provides for the analysis of the sample solution in the isolated wells after amplification. In a preferred embodiment, the invention includes the steps of detecting an amount of a first target nucleic acid sequence in said fractionated liquid sample; detecting an amount of a second target nucleic acid sequence in said fractionated liquid sample; and, determining whether a difference exists between the amounts of said first target nucleic acid sequences and said second target nucleic sequences. The presence of a statistically-significant difference being indicative of a presence of disease in a patient from whom said liquid sample is obtained. An embodiment of the invention further includes the use of labels on target nucleic acid sequence with a detectable label, such as, for example, radioisotopes, fluorescent compounds, and enzymatic markers. Other embodiments of the invention include the steps of performing assays on the sample such as, for example, a multiple mutation assay, a Bat-26 assay, an assay to detect loss of heterozygosity, and the like.

In additional embodiments, after the detection and/or analysis of the nucleic acids, the invention includes the steps of performing a colonoscopy or a sigmoidoscopy on a patient from whom a liquid sample is obtained and analyzed.

Equivalents

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

Incorporation by Reference

All patents, patent applications, and scientific publications mentioned herein above are incorporated by reference into this application in their entirety.

We claim:

1. A device for separating a liquid sample, the device comprising:
a first layer slidably attached to a second layer, said first layer comprising a first well, and said second layer comprising a second well, wherein said first well is in fluid communication with said second well when said layers are in a first position; and,
wherein said first well in said first layer is isolated from said second well in said second layer when said layers are in a second position.

2. The device of claim 1, wherein a plurality of wells in said first and said second layers provide a pathway of concatenated wells when said layers are in said first position.

3. The device of claim 2, further comprising a plurality of pathways of concatenated wells.

4. The device of claim 3, wherein at least one of said pathways of concatenated wells comprises a liquid sample, and at least one of said pathways of concatenated wells comprises a detectable polynucleotide.

5. The device of claim 3, wherein at least one of said pathways of concatenated wells comprises a liquid sample, and at least one of said pathways of concatenated wells comprises a molecular beacon.

6. The device of claim 1, further comprising a fill port for introducing said liquid sample into the device.

7. The device of claim 1, further comprising a first system providing a vacuum in the device to draw said liquid sample from said first well to said second well.

8. The device of claim 7, wherein said first system comprises a sample suction port connected to said pathway of concatenated wells whereby movement of said liquid sample through said pathway of concatenated wells is driven by a vacuum applied to said sample suction port.

9. The device of claim 7, further comprising a valve actuated at a higher pressure than said vacuum provided by said first system, thereby to prevent said liquid sample from evacuating said device.

10. The device of claim 6, further comprising a reservoir connected to said fill port for providing said liquid sample to said device.

11. The device of claim 1, further comprising a second system providing a vacuum line in the device whereby said first and said second layers are secured to prevent leakage of sample.

12. The device of claim 11, wherein said second system comprises a plurality of vacuum lines.

13. The device of claim 12, wherein said second system comprises a vacuum port connected to said vacuum line.

14. The device of claim 1, whereby said first and second layers are lubricated with a lubricant substantially promoting a seal between said first and second layers.

15. The device of claim 1, wherein said first and second wells are etched, machined, stamped, or embossed onto a substrate.

16. The device of claim 7, wherein said first system providing a vacuum is etched, machined, stamped, or embossed onto a substrate.

17. The device of claim 11, wherein said second system providing a vacuum is etched, machined, stamped, or embossed onto a substrate.

18. The device of claim 1, wherein said first layer and said second layer are identical.

19. A device for separating a liquid sample, the device comprising:
a fill port for introducing a liquid sample into the device;
a first layer slidably attached to a second layer, said first and second layer comprising;
at least two wells having a first position wherein a first well is in fluid communication with a second well; and,
said at least two wells having a second position wherein said first well is isolated from said second well;
a first system providing a vacuum to actuate movement of said liquid sample from said first well to said second well; and
a second system providing a vacuum line in said device to secure said first and said second layers and to substantially prevent said liquid sample from evacuating said device.

20. A method for separating a liquid sample, the method comprising the steps of:
introducing a liquid sample into a device according to claim 1; and
providing a vacuum in said device thereby to move of said liquid sample through said first well into said second well; and
sliding said device from said first position to said second position thereby separating said liquid sample into subsamples in said first well and said second well.

21. The method of claim 20, wherein said liquid sample substantially fills said first well and said second well prior to said sliding step.

22. The method of claim 20, further comprising a plurality of wells in fluid communication with said second well providing a pathway of concatenated wells.

23. The method of claim 20, wherein said liquid sample substantially fills said pathway of concatenated wells prior to said sliding step.

24. The method of claim 20, wherein said step of providing a vacuum in said device substantially removes the presence of air in said device prior to said sliding step.

25. The method of claim 20, wherein said sample is a biological sample.

26. The method of claim 20, further comprising the step of analyzing said liquid subsamples.

27. The method of claim 26, wherein said analyzing step comprises conducting a polymerase chain reaction.

28. The method of claim 26, wherein said analyzing step further comprises the steps of:
a) detecting an amount of a first target nucleic acid sequence in said fractionated liquid sample;
b) detecting an amount of a second target nucleic acid sequence in said fractionated liquid sample; and
c) determining whether a difference exists between the amounts of said first target nucleic acid sequences and said second target nucleic sequences;
the presence of a statistically-significant difference being indicative of a presence of disease in a patient from whom said liquid sample is obtained.

29. The method of claim 26, wherein said analyzing step includes optical imaging and analysis.

30. The method of claim 26, further comprising the step of labeling a target nucleic acid sequence with a detectable label.

31. The method of claim 30, wherein said detectable label is selected from the group consisting of radioisotopes, fluorescent compounds, and enzymatic markers.

32. The method of claim 20, further comprising the step of performing a colonoscopy on a patient from whom said liquid sample is obtained.

33. The method of claim 20, further comprising the step of performing a sigmoidoscopy on a patient from whom said liquid sample is obtained
